(19) United States
(12) Patent Application Publication
(10) Pub. No.: US 2007/0225579 A1
(43) Pub. Date: Sep. 27, 2007

(54) Fiber Optic Probe

(75) Inventors: Gerard Lucassen, Eindhoven (NL); Geert Gijsbers, Best (NL); Michael Cornelis Van Beek, Eindhoven (NL); Sieglinde Neerken, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/568,995

(22) PCT Filed: May 9, 2005
(86) PCT No.: PCT/IB05/51498

§ 371 Date: Nov. 13, 2006

(30) Foreign Application Priority Data
May 14, 2004 (EP) 04102115.5

(51) Int. Cl. A61B 5/1455

(52) U.S. Cl. 600/310

(57) ABSTRACT

The present invention provides a spectroscopic system and in particular a fiber optic probe for in vivo detection of vulnerable atherosclerotic plaque inside the cardiovascular system of a patient. Detection of vulnerable plaque is based on a location dependent measurement of concentration levels of cardiac marker molecules flowing in the blood stream. Concentration level determination is preferably based on surface-enhanced Raman spectroscopic techniques providing sufficient sensitivity for determination of these concentration levels. Acquisition of spectroscopic data during moving of the fiber optic probe through the cardiovascular system with controlled velocity allows to precisely locate vulnerable plaque in the cardiovascular system.

The present invention relates to the field of spectroscopy and in particular without limitation to a fiber optic probe for spectroscopy analysis.

The acute myocardial infarction, commonly denoted as heart attack, is a frequent cause of death. An underlying mechanism of a heart attack is the destruction of heart muscle cells due to a lack of oxygen. Obstruction of a coronary artery is probably the most prominent reason for such a lack of oxygen and triggers myocardial infarction. A life threatening obstruction of a coronary artery very often arises due to atherosclerosis. Typically, atherosclerosis results in the generation of vulnerable plaque that is formed by absorption of fat droplets by an artery. Resident cells of an artery wall interpret such an absorption as an intrusion that initiates a release of proteins in form of cytokines that lead to inflammation. The cytokines make the artery wall sticky and immune system cells, e.g. monocytes are attracted. The monocytes circulating in the blood enter the artery wall, turn into macrophages and ingest the fat droplets or LDL particles. Thereby, the macrophages turn into large fat filled cells that form vulnerable plaque with a thin covering denoted as fibrous cap.

Deposition of vulnerable plaque covered by fibrous cap at arterial walls on the one hand narrows the blood vessel leading to a narrowing of the artery. On the other hand these fatty deposits are rather fragile and the fibrous cap is prone to crack or rupture. In case the fibrous cap is subject to rupture, the content of the vulnerable plaque is spilled into the blood stream. As a result a blood clot may form around the rupture leading to a partial blockage or even a complete obstruction of the artery.

It is therefore necessary to provide diagnostic techniques for identifying plaques that are prone to disruption. Identification of these potentially lethal plaques before they disrupt will facilitate the development of the therapeutic strategies or prevent acute events.

The patent application US 2001/0047137 A1 discloses methods and apparatus for detecting and analyzing the composition of vulnerable plaques in living tissue with near infrared (NIR) radiation. Identification of plaques with a lipid pool and thin cap in vivo may be performed through a NIR coronary catheter for identifying vulnerable plaques in the coronary arteries of living patients. A fiber optic probe may be operatively connected to a light source, and a light directing or focusing mechanism may be mounted to the distal end of the probe. The focusing mechanism may comprise a compound parabolic concentrator (CPC) that may be adapted to compress the incident beam from the transmitting fiber optic onto a small spot on the tissue surface undergoing analysis.

The apparatus may include detectors such as lead sulfide detectors for detecting the scattered light from the artery surface or other tissue being analyzed. Further, the fiber optic probe may be preferably adapted for introduction into a patient to thereby allow in vivo analysis of artery walls, or in particular, lesions which may characterized as vulnerable plaques. The disclosed method includes the steps of focusing light on the tissue to be analyzed, and detecting light reflected by the tissue.

Prior art solutions for identification and characterization of vulnerable atherosclerotic plaques based on near infrared spectroscopy are designed for focusing NIR radiation onto the wall of a blood vessel. Therefore, appropriate focusing mechanisms are always required.

The present invention provides a fiber optic probe of a spectroscopic system that comprises means for directing an excitation radiation into a volume of interest, means for collecting return radiation from the volume of interest and means for inducing a surface-enhanced spectroscopic effect. The inventive fiber optic probe is adapted to be coupled to a light source providing radiation in the near infrared range. Typically, the fiber optic probe is adapted for emission of the near infrared radiation as excitation radiation into a volume of interest. The inventive fiber optic probe is furthermore designed for intravascular and in vivo detection and analysis of vulnerable plaque. The volume of interest typically specifies a volume entirely located within a cardiovascular system of a patient.

The fiber optic probe may comprise a bundle of optical fibers that allow for emission of excitation radiation as well as for collection of return radiation emanating from the volume of interest as a product of scattering processes of the excitation radiation within the volume of interest. The optical fibers for collecting return radiation also provide transmission of the return radiation to a spectroscopic apparatus of a spectroscopic system for spectroscopic analysis of the collected return radiation. The spectroscopic analysis of the return radiation allows for a precise analysis of biological structures and/or substances that are located within the volume of interest.

At its end facet the fiber optic probe may comprise an objective lens providing efficient emission of excitation radiation as well as efficient collection of return radiation. Moreover, the means for inducing a surface-enhanced spectroscopic effect provide a significant signal enhancement of the return radiation. In this way the sensitivity of the spectroscopic system can be appreciably enhanced. Within the framework of spectroscopy and in particular in Raman spectroscopy, surface-enhanced effects like surface-enhanced Raman spectroscopy (SERS) even provide detection of single molecules.

Generally, the rather weak Raman effect can be greatly strengthened if the molecules that are subject to Raman scattering are attached to nanometer sized noble metal structures. When a scattering molecule is in close proximity to a metal surface, an enhancement in Raman intensity arises from coherent superposition of the incident and reflected fields at the position of the molecule and due to excitation of surface plasmons by electromagnetic radiation. The means for inducing the surface-enhanced spectroscopic effect are preferably designed for surface-enhanced Raman spectroscopy. Consequently, the fiber optic probe features a high sensitivity and the spectroscopic system may be even adapted to detect single molecules within the volume of interest.

According to a further preferred embodiment of the invention, the means for inducing the surface-enhanced spectroscopic effect comprise a thin metal coating having a thickness in the range of nanometers. Practically, the end facet of the fiber optic probe that is adapted for emission as well as for detection of excitation and return radiation, respectively, is coated with the thin metal coating. Generally, each metal for which SERS observations have been reported can be used. This includes the noble metals, for which SERS has been well characterized, the alkali metals, Al and In.

Preferably, the distal end of the fiber optic probe is coated with a thin layer of gold. In this way radiation that is due to an inelastic scattering process taking place in close proximity to the metal coating is appreciably enhanced. Making use of SERS techniques, the volume of interest is located in close proximity to the surface of the thin metal coating. Hence, a focusing of the excitation radiation into a designated substance or biological structure, like. e.g. tissue, is in principle not required. The volume of interest defines shell encompassing the metal coating.

According to a further preferred embodiment of the invention, the thin metal coating is further adapted to absorb marker molecules. Marker molecules and in particular cardiac markers are typically released in the blood stream of a patient as phase reactants in response to inflammation that might be due to deposition of vulnerable plaque. Increased levels of cardiac markers in the blood stream of a patient have been shown to predict cardiovascular events. Absorbing and hence accumulating cardiac marker molecules at the thin metal coating is an effective means to determine a concentration level of cardiac markers within a blood stream by making use of SERS.

Once being absorbed at the thin metal coating, the cardiac marker molecules are in such a close proximity to the surface of the metal coating that surface-enhanced spectroscopic effects may evolve when the cardiac marker molecule is subject to an inelastic scattering process.

There exists a plurality of various substances that are known to act as cardiac markers. These are for example troponin 1 (Tn1), troponin T (TnT), troponin C (TnC), myoglobin, fatty acid binding protein (FABP), Glycogen Phosphorylase Isoenzyme BB (GPBB), high sensitivity CRP, etc. . . Elevated concentrations of any one of these cardiac markers indicate cardiac tissue injury or even myocardial infarction. The high sensitivity C-reactive protein (hsCRP) is known for several decades as a non-specific inflammation marker. In particular, high CRP levels are detected in human blood during bacterial, viral and other infections. Among other markers of inflammation, CRP and IL-6 show the strongest association with cardiovascular events. CRP levels can increase to as much as 1-1000 fold from base line concentrations due to inflammatory events, such as deposition of vulnerable plaque at a vessel wall.

Preferably, the thin metal coating of the fiber optic probe is prepared with capture molecules that are capable of capturing specific cardiac markers. The capture molecules are therefore immobilized on the metal coating of the fiber optic probe. Capture molecules that are adapted for capturing of e.g. hsCRP or CRP marker molecules are for example monoclonal antibodies to hsCRP, CRP antigen and CRP free serum. These and various other types of antibodies that are designed for capturing of cardiac markers are commercially available from e.g. Hytest Ltd. in Turku, Finland, for further information on cardiac marker also refer to http://www.hytest.fi .

According to a further preferred embodiment of the invention, the thin metal coating further comprises a surface roughness for absorbing of the marker molecules. Designing the thin metal coating with a distinct surface roughness on the one hand provides an enhanced adhesion of capture molecules for the cardiac markers and therefore an improved absorption of cardiac marker molecules to the fiber optic probe. On the other hand, a rough surface of the thin metal coating is also advantageous for the surface enhancing spectroscopic effect. A roughened surface of the thin metal coating is particularly advantageous for the excitation of surface plasmons resulting in enhanced electromagnetic fields close to the surface. Compared to a smooth surface, the roughened surface also features a slightly increased total area which positively influences the sensitivity of the fiber optic probe.

According to a further preferred embodiment of the invention, the fiber optic probe is designed as a catheter that is adapted to be inserted into a vascular system of a patient. Hence, the fiber optic probe can be effectively used for intravascular examination of a patient. Levels of concentration of cardiac markers can be precisely determined in vivo in a minimal invasive way. Since the fiber optic probe can be adapted for detection of a particular cardiac marker molecule, such as CRP, a spectroscopic analysis of surface-enhanced Raman signals may be performed almost instantaneously. Hence a direct monitoring of cardiac marker levels within a blood stream can be realized allowing for a precise and fast diagnosis of a level of inflammation of an e.g. arterial wall.

According to a further preferred embodiment of the invention, the fiber optic probe is further adapted to be moved through the vascular system of the patient. Inserting the distal end of the fiber optic probe as a catheter into the vascular system of the patient and moving the distal end of the fiber optic probe through the vascular system of the patient allows to monitor a level of cardiac marker concentration in dependence of the location of the fiber optic probe within the cardiovascular system of the patient. In particular, by pulling back of the fiber optic probe with a constant velocity, that is typically in the range of e.g. less than a millimeter per minute, a location dependent concentration distribution of cardiac markers can be obtained. Further assuming that the level of cardiac marker concentration appreciably increases in the proximity of inflamed tissue, the position of vulnerable plaque inside a cardiovascular system can be precisely determined. Pulling back of the fiber optic probe is by no means restricted to a movement with constant velocity. Moreover, the fiber optic probe can be pulled back with varying speed. In this case, analysis of collected return radiation has to account for the velocity of movement.

The invention therefore provides an effective means for detecting vulnerable plaque in a cardiovascular system of a patient in vivo by making use of a spatially resolved measurement of cardiac marker concentration within the blood stream. Measurement of concentration level of cardiac marker molecules is performed by means of a surface-enhanced spectroscopic effect such as SERS that provides a sensitivity that is sufficient for a precise determination of the relevant concentration levels.

According to a further preferred embodiment of the invention, the fiber optic probe comprises a reflective element that is further adapted as means for inducing the surface-enhanced spectroscopic effect. Preferably, the reflective element features a metal coating with a roughened surface that is ideally suited for adsorption of cardiac marker molecules. Additionally, the reflective element features a high reflectivity for both excitation as well as frequency shifted return radiation. The reflective element serves as a reflector for emitted radiation in order to increase the ratio of the intensities of return to excitation radiation. Therefore, the reflective element is preferably arranged opposite to the end facet of the fiber optic probe or slanted at an angle with respect to the normal of the end facet. The reflective element may be implemented as a mirror element, such as e.g. a spherical or parabolic mirror. Adhesion of cardiac marker molecules at the reflective element therefore provides an effective enhancement of the intensity of the return radiation.

In another aspect, the invention provides a spectroscopic system comprising a fiber optic probe for directing excitation radiation into a volume of interest and for collecting return radiation from the volume of interest. Herein, the fiber optic probe is coated with a thin metal coating at its distal end that serves to induce a surface-enhanced spectroscopic effect. The spectroscopic system further comprises a spectroscopic apparatus that is adapted to spectrally analyze the collected return radiation and that is adapted to be connected to a proximal end of the fiber optical probe.

Preferably, the fiber optic probe and the spectroscopic apparatus are connected by a single or by a plurality of optical fibers providing bi-directional transmission of excitation radiation and return radiation between the fiber optic probe and the spectroscopic apparatus. The spectroscopic apparatus comprises a light source generating the near infrared excitation radiation. In this way the fiber optic probe can be designed in a compact way allowing for insertion of the fiber optic probe into a cardiovascular system of a patient as a catheter. Components such as power supply, spectral analysis unit, light source as well as signal processing means are typically provided by the spectroscopic apparatus.

According to a further preferred embodiment of the invention, the spectroscopic system further comprises means for moving the distal end of the fiber optical probe through the vascular system of the patient. In this embodiment the spectroscopic system further comprises means for correlating the collected return radiation with a location of the distal end of the fiber optic probe. Since the spectroscopic system is adapted to determine a concentration level of distinct cardiac marker molecules in a blood stream, moving of the distal end of the fiber optical probe in combination with collecting scattered return radiation provides a spatially resolved concentration level of the cardiac markers along a blood vessel. Determining a longitudinal location of the distal end of the fiber optical probe within the vascular system of the patient and monitoring corresponding spectroscopic signals allows to correlate the location of the optical probe with collected return radiation. Since the return radiation is indicative of the concentration level of cardiac markers, the location of vulnerable plaque featuring a high concentration of cardiac markers in its proximity can be precisely determined.

In another aspect, the invention provides a spectroscopic apparatus of a spectroscopic system, wherein the spectroscopic apparatus comprises coupling means for connecting the spectroscopic apparatus with a proximal end of a fiber optic probe, analyzing means that are adapted to spectrally analyze return radiation that is collected by the fiber optic probe and correlation means that are adapted to correlate the collected return radiation with a location of the distal end of the fiber optic probe. The fiber optic probe is coated with a thin metal coating for inducing a surface-enhanced spectroscopic effect.

The fiber optic probe is furthermore implemented as a catheter that can be inserted into a vascular system of a patient. Spectral analysis of the collected return radiation is indicative of cardiac marker level concentration within the blood stream of the patient. By correlating the spectrally analyzed return radiation with the location of the distal end of the fiber optic probe within the vascular system of the patient, inflammations such as vulnerable plaque can be precisely determined and localized in vivo in a minimal invasive way.

In another aspect, the invention provides a computer program product for a spectroscopic system having a fiber optic probe and a spectroscopic apparatus. The computer program product comprises computer program means that are adapted for moving of a distal end of the fiber optic probe through a vascular system of a patient, determining the location of the distal end of the fiber optic probe and for correlating the location of the distal end of the fiber optic probe with collected return radiation obtained from the fiber optic probe. Hence, the computer program product is adapted for generating and eventually visualizing a spatially resolved measurement of concentration of cardiac markers in the blood stream of the patient.

In still another aspect, the invention provides a method of determining of cardiac marker concentration within a cardiovascular system of a patient. The method comprises of inserting a distal end of a fiber optic probe being designed as a catheter into the cardiovascular system of the patient. The distal end of the fiber optic probe is adapted for collecting return radiation from a volume of interest and is further adapted to induce a surface-enhanced spectroscopic effect, e.g. SERS. The method further comprises spectrally analyzing the collected return radiation and moving the distal end of the catheter during collection of the return radiation. Finally the method provides a correlation step wherein the location of the distal end of the fiber optic probe is correlated with the return radiation and/or spectrally analyzed return radiation from corresponding locations of the fiber optic probe.

In the following preferred embodiments of the invention will be described in detail by making reference to the drawing in which:

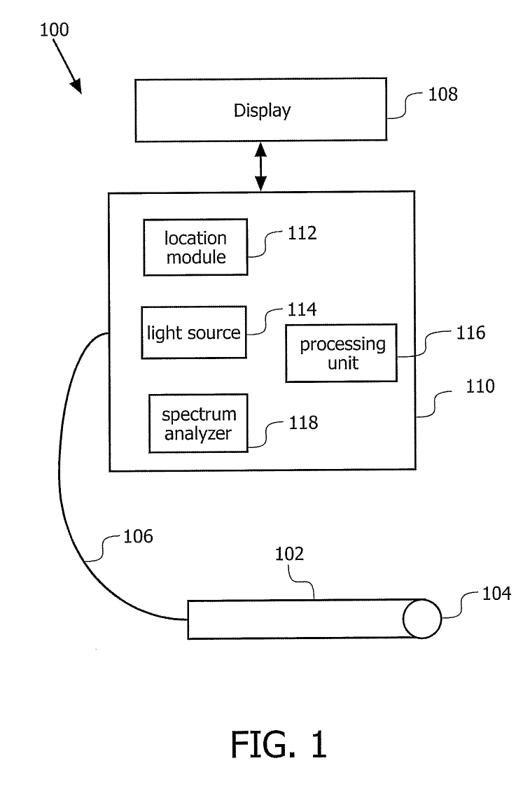

FIG. 1 is illustrative of a block diagram of the spectroscopic system,

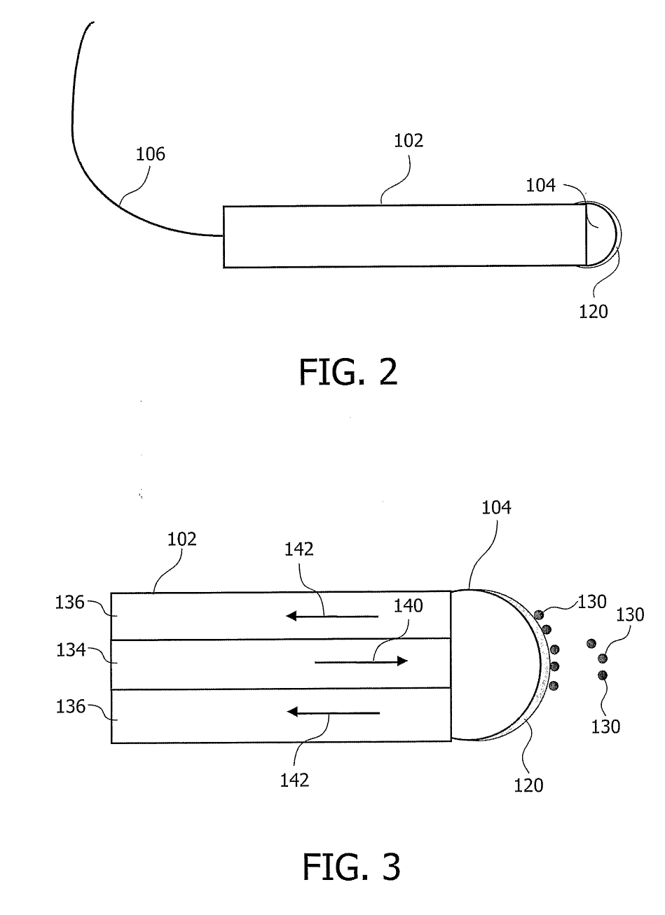

FIG. 2 shows a block diagram of the fiber optic probe,

FIG. 3 shows a cross sectional illustration of the fiber optic probe with adsorbed cardiac marker molecules,

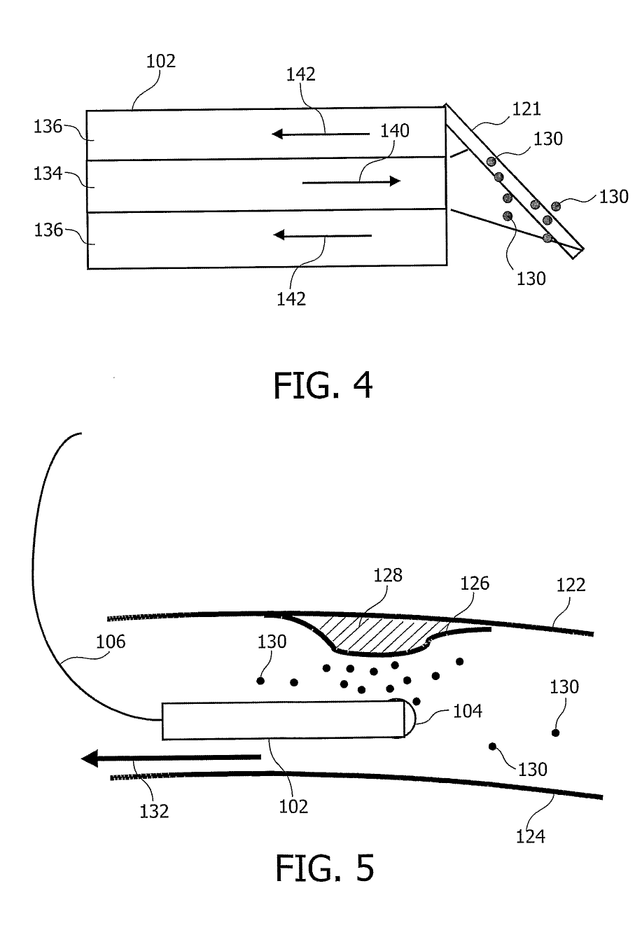

FIG. 4 shows a fiber optic probe with are reflector,

FIG. 5 shows a cross sectional illustration of the fiber optic probe inserted in a cardiovascular system,

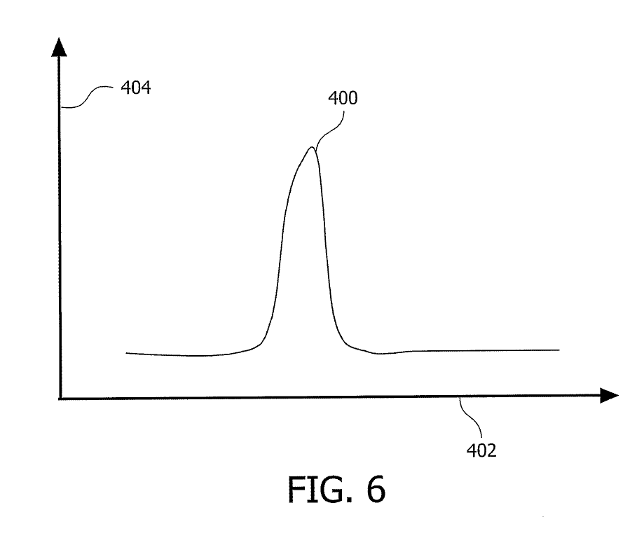

FIG. 6 shows a diagram illustrating the cardiac marker concentration versus position of the probe head within a blood vessel.

FIG. 1 shows a block diagram of a spectroscopic system 100 comprising a fiber optic probe 102, an optical fiber 106, a spectroscopic apparatus 110 and a display 108. The fiber optic probe 102 has an objective lens 104 and is connected to the spectroscopic apparatus 110 via the optical fiber 106.

The spectroscopic apparatus 110 has a location module 112, a light source 114, a spectrum analyzer 118 and a processing unit 116. The spectroscopic apparatus 110 is connected with the display 108 for providing analyzed data to a user of the spectroscopic system 100. The fiber optic probe 102 is preferably adapted to be inserted into a cardiovascular system of a patient. This requires a compact design of the fiber optic probe 102 and therefore voluminous components of the spectroscopic system like light source 114 and spectrum analyzer 118 are located within the spectroscopic apparatus 110.

The fiber optic probe 102 serves to emit excitation radiation into a volume of interest and for collecting in elastically scattered return radiation that is frequency shifted with respect to the excitation radiation and therefore indicative of the molecular composition of the volume of interest. In the illustrated embodiment, excitation radiation in the near infrared range is generated by the light source 114 and transmitted to the fiber optic probe 102 via the optical fiber 106. By means of the objective lens 104 the excitation radiation is focused into the volume of interest. For the present invention, the excitation radiation does not necessarily have to be focused. The objective lens 104 does therefore not represent an essential component of the fiber optic probe 102. In principle, the fiber optic probe 102 only has to support emission of excitation radiation into the volume of interest and collection of return radiation. Additionally, the fiber optic probe 102 has to provide efficient coupling of excitation radiation and return radiation from and into the optical fiber 106.

The fiber optic probe 102 and the objective lens 104 are particularly designed for detection of surface-enhanced Raman spectroscopic signals emanating from designated cardiac marker molecules within a blood stream. In particular by making use of SERS the sensitivity of the spectroscopic system 100 is appreciably increased. Spectroscopic techniques based on SERS in principle allow for detection of single molecules. Surface-enhanced spectroscopic signals emanating from cardiac marker molecules that are in close proximity to a metal coating of the fiber optic probe 102 are collected by the fiber optic probe 102 and transmitted to the spectroscopic apparatus 110. There, the surface-enhanced spectroscopic signals are analyzed by means of the spectrum analyzer 118 and further processed by the processing unit 116.

Since the fiber optic probe is implemented as a moveable catheter, by means of the location module 112 the location of the fiber optic probe 102 within the cardiovascular system of the patient has to be determined. Location information of the fiber optic probe 102 can then be correlated with analyzed spectroscopic signals obtained from corresponding locations in order to generate a diagram illustrating cardiac marker concentration versus position of the fiber optic probe 102. Correlation of location information and corresponding spectroscopic signals being indicative of cardiac marker concentration is performed by the processing unit 116. Preferably, the processing unit 116 makes use of a computer program product for correlating the location information obtained from the location module 112 with spectroscopic data obtained from the spectrum analyzer 118. Moreover, the processing unit 116 may provide a graphical illustration of the acquired and correlated data by making use of the display 108.

Moreover, the spectroscopic system 100 comprises means for moving of the fiber optic probe 102 through the cardiovascular system of the patient. This preferably constant movement is performed in a pull back mode, i.e. the fiber optic probe 102 is inserted into a blood vessel up to a certain position and is then successively pulled back with a constant velocity. During this constant movement spectroscopic data is acquired. Alternatively, a movement with variable speed can be performed allowing for a more flexible handling of the catheter.

FIG. 2 shows a block diagram of the fiber optic probe 102 with an objective lens 104 and an optical fiber 106. Here, fiber optic probe 102, objective lens 104 and optical fiber 106 provide the same functionality as already illustrated in FIG. 1. Additionally, the distal end of 104 of the fiber optic probe 102 comprises a thin metal coating that serves to induce a surface-enhanced spectroscopic effect like surface-enhanced Raman spectroscopy (SERS). Typically, the thickness of the metal coating is in the range of a few nanometers and features a distinct surface roughness. The thickness and the roughness of the thin metal coating are designed for optimum enhancement of electromagnetic fields close to the surface of the thin metal coating 120.

FIG. 3 shows a cross sectional illustration of the fiber optic probe 102. Here, the fiber optic probe 102 comprises two fiber optic waveguides 134 and 136. Waveguide 134 is implemented as a core of an optical fiber for transmission of excitation radiation 140, whereas waveguide 136 serves as a multi-mode cladding for transmission of return radiation 142. In principle, the inner waveguide 134 may be implemented as a single-mode waveguide whereas the waveguide 136 is preferably implemented as a multi-mode wave guiding structure. Alternatively, both wave guiding structures 134, 136 may be designed as bundle of optical fibers.

In this embodiment, the objective lens 104 provides emission of the excitation radiation 140 as well as collection of the return radiation 142. Appropriate filters embedded at the end facet of the fiber 102 may provide sufficient spectral filtering of the collected radiation.

The objective lens 102 has a coating 120 of preferably noble metal to induce a surface-enhanced spectroscopic effect. Moreover, since surface-enhanced Raman spectroscopy requires that inelastic scattering processes occur in close proximity to the surface of the thin metal coating 120, the metal coating is preferably subject to surface treatment. By means of such a surface treatment specific capture molecules are prepared in such a way that they are immobilized on the metal coating and that they are capable of capturing of specific cardiac markers 130, such as CRP or hsCRP, flowing in the blood stream. This is to ensure that the distance of the cardiac markers 130 to the metal coating 120 is below a certain threshold allowing for sufficient signal enhancement. Typically, this threshold is in the range of several tens of nanometers.

Capture molecules 130 are for example monoclonal antibodies to various cardiac markers like troponin 1 (Tn1), troponin T (TnT), troponin C (TnC), myoglobin, fatty acid binding protein (FABP), Glycogen Phosphorylase Isoenzyme BB (GPBB), high sensitivity CRP, etc. . . .

FIG. 4 is illustrative of a fiber optic probe 102 with a reflective element 121. Similar as illustrated in FIG. 3, the fiber optic probe 102 has a plurality of waveguides 134 and 136 that provide sufficient counter-directional propagation of excitation 140 and return radiation 142. Here, the fiber optic probe 102 does not make use of an objective lens. Instead the end facet of the fiber optic probe 102 has a reflective element 121 that features a high reflectivity for the excitation as well as for the return radiation. Excitation radiation 140 emitting from the waveguide 134 is reflected at the surface of the reflective element 121 and re-enters the waveguide 142. During this propagation the excitation radiation may become subject to an inelastic scattering process with a cardiac marker molecule 130. Resulting frequency-shifted return radiation may then be collected by the waveguide 136 and transmitted to a spectrum analyzer.

In order to enhance the probability of inelastic scattering processes to occur, the reflective element is preferably coated with the thin metal coating that induces the surface-enhanced spectroscopic effect. Moreover, the reflective element 121 has been subject to a surface treatment process in order to immobilize distinct capture molecules at its surface. In this way, the reflective element is adapted to aggregate an to adsorb a distinct type of cardiac marker molecules. As a result the signal ratio of excitation to return radiation may appreciably enhance.

Generally, a large variety of embodiments making use of a reflective element are conceivable. The slanted reflective element 121 in FIG. 4 is one example. Other types of reflective elements like e.g. spherical or parabolic mirrors can also be implemented that may even further enhance the intensity of the return radiation.

FIG. 5 shows a cross sectional illustration of the inventive fiber optic probe 102 when inserted into a cardiovascular system of a patient. Vessel walls 122, 124 illustrate upper and lower boundary of a blood vessel. The upper part of the vessel wall 122 is subject to atherosclerosis as can be seen by the deposition of vulnerable plaque 128 covered by a fibrous cap 126. The plurality of cardiac marker molecules 130 is indicative of the inflammation represented by the vulnerable plaque 128 and the fibrous cap 126. Cardiac markers such as C-reactive protein (CRP) or high sensitivity C-reactive protein (hsCRP) are acute phase reactants and are released by the liver in response to acute injury, infection, or other inflammatory stimuli. Cardiac markers like CRP aggregate near the inflammation or lesion represented by the vulnerable plaque 128. Therefore in the proximity of the vulnerable plaque 128 the concentration of CRP is appreciably higher than in regions that are not subject to inflammation.

Pulling the fiber optic probe 102 to the left as indicated by the pull direction 132 while simultaneously monitoring surface-enhanced Raman spectroscopic signals emanating from the cardiac marker molecules 130 allows to generate a location specific cardiac marker distribution.

FIG. 6 is illustrative of a diagram providing a concentration of cardiac markers versus position of the fiber optic probe. In the vertical direction 404 the diagram provides concentration levels of a designated cardiac marker molecule. In the horizontal direction 402 the longitudinal position of the fiber optic probe within a blood vessel is given. Plotting of the concentration levels of cardiac marker molecules versus position of the fiber optic probe results in the graph 400. The graph 400 features a remarkable peak at a certain distance indicating the position of an inflammation within the blood vessel, such as e.g. absorption of vulnerable plaque. The height of the peak in turn indicates the level of concentration of the cardiac markers and is therefore indicative of the level of inflammation.

By making use of a variety of different cardiac markers that particularly differ with respect to the spectroscopic properties different kinds of inflammation may be sufficiently detected.

### LIST OF REFERENCE NUMERALS

100 spectroscopic system
102 fiber optic probe
104 objective lens
106 optical fiber
108 display
110 spectroscopic apparatus
112 location module
114 light source
116 processing unit
118 spectrum analyzer
120 coating
121 reflector
122 vessel wall
124 vessel wall
126 fibrous cap
128 plaque
130 cardiac marker molecule
132 pull direction
134 waveguide
136 waveguide
140 excitation radiation
142 return radiation

1. A fiber optic probe (102) of a spectroscopic system comprising:
means for directing excitation radiation (140)into a volume of interest,
means for collecting return radiation (142) from the volume of interest,
means for inducing a surface-enhanced spectroscopic effect.

2. The fiber optic (102) probe according to claim 1, wherein the means for inducing the surface-enhanced spectroscopic effect comprising a thin metal coating (120), the thickness of the metal coating being in a range of nanometers.

3. The fiber optic probe (102) according to claim 2, wherein the thin metal coating (120) being adapted to adsorb marker molecules.

4. The fiber optic probe (102) according to claim 3, wherein the thin metal coating (120) further comprising a surface roughness being adapted for adsorbing of the marker molecules (130).

5. The fiber optic probe (102) according to claim 1, wherein the fiber optic probe being designed as a catheter being adapted to be inserted into a vascular system of a patient.

6. The fiber optic probe (102) according to claim 5, being further adapted to be moved through the vascular system of the patient.

7. The fiber optic probe (102) according to claim 1, further comprising a reflective element (121) being adapted as means for inducing the surface-enhanced spectroscopic effect.

8. A spectroscopic system (100) comprising:
a fiber optic probe (102) for directing excitation radiation (140) into a volume of interest and for collecting return radiation (142) from the volume of interest, wherein the fiber optic probe being coated with a thin metal coating (120) at its distal end,
a spectroscopic apparatus (110) being adapted to spectrally analyze the collected return radiation (142) and being adapted to be connected to a proximal end of the fiber optic probe (102).

9. The spectroscopic system (100) according to claim 8, further comprising:
means for moving the distal end of the fiber optic probe (102) through a vascular system of a patient,
means for correlating the collected return radiation (142) with a location of the distal end of the fiber optic probe (102).

10. A spectroscopic apparatus (110) of a spectroscopic system (100) having a fiber optic probe (102) for directing excitation radiation (140) into a volume of interest and for collecting return radiation (142) from the volume of interest, wherein the fiber optic probe being coated with a thin metal coating (120) at its distal end, the spectroscopic apparatus comprising:
coupling means for connecting the spectroscopic apparatus (110) with the proximal end of the fiber optic probe (102),
analyzing means being adapted to spectrally analyze the collected return radiation (142),
correlation means being adapted to correlate the collected return radiation with a location of the distal end of the fiber optic probe.

11. A computer program product for a spectroscopic system (100) having a fiber optic probe (102) and a spectroscopic apparatus (110), the computer program product comprising computer program means being adapted for:
moving of a distal end of the fiber optic probe (102) through a vascular system of a patient,
determining the location of the distal end of the fiber optic probe,
correlating the location of the distal end of the fiber optic probe with the collected return radiation (142).

12. A method of determining of cardiac marker concentration within a cardiovascular system of a patient, the method comprising the steps of:
collecting return radiation (142) from a volume of interest by a distal end of a fiber optic probe (102)
inducing a surface-enhanced spectroscopic effect by the distal end of a fiber optic probe (102),
spectrally analyzing the collected return radiation (142),
moving the distal end of the catheter during collection of return radiation,
correlating a location of the distal end of the fiber optic probe with the collected return radiation.
